(19) United States
(12) Patent Application Publication
(10) Pub. No.: US 2019/0096531 A1
(43) Pub. Date: Mar. 28, 2019

(54) SYSTEM AND METHOD OF PREVENTIVE HEALTH MANAGEMENT FOR ASSESSING AN INDIVIDUAL'S HEALTH THROUGH CONSOLIDATION AND DIGITIZATION OF MEDICAL RECORDS

(71) Applicant: AAYUV TECHNOLOGIES PRIVATE LIMITED, India (IN)

(72) Inventors: Kiran Kalakuntla, Hyderabad (IN); Srikanth Samudrala, Hydearabad (IN)

(21) Appl. No.: 16/082,945

(22) PCT Filed: Mar. 15, 2017
(86) PCT No.: PCT/IB2017/051501

§ 371 Date: Sep. 7, 2018

(30) Foreign Application Priority Data
Mar. 16, 2016 (IN) 201641009211

(51) Int. Cl. G16H 50/70; G16H 50/20; G16H 10/60; G16H 40/60

(52) U.S. Cl. CPC G16H 50/70; G16H 40/60; G16H 10/60; G16H 50/20

(57) ABSTRACT

Exemplary embodiments are directed towards a system and method for preventive health management for assessing an individual's health through consolidation and digitization of medical records. The system includes a medical history consolidating module configured to consolidate medical records of an individual in a digital format and an assessment module configured to analyse medical records of an individual. The system further includes a display module configured for interactively displaying consolidated medical records of the individual in response to analysing the medical records. The interactive displaying of health records enables an individual to self-learn and self-understand health profile.

### TECHNICAL FIELD

The present disclosure generally relates to the field of maintaining medical records management. More particularly, the present disclosure relates to a system and method for assessing an individual's health through consolidation and digitization of medical records.

### BACKGROUND

Medical records maintenance have always been the prerogative of the health services provider as they would store and keep a track of the records associated with an individual. Personalising the medical or medical records has been relatively recent. Off late individuals have started realising the importance of maintaining one's medical records in order to maintain better health. Given the mechanical lifestyle it is a mammoth task to go around looking for a health care provider, obtaining an appointment, visiting them and going ahead with the further prescribed procedures.

A timely assessment of potential health risks may slow down their adverse impact on an individual's health thus buying time for recovery. Increased health awareness would motivate an individual to go ahead with improvised health care plans. It would be very easy and effective when an individual's medical records are made available on a single platform with rapid analysis and which is easily comprehendible.

There are several reasons for the lack of a centralized database. First being the importance of maintaining the privacy of data. It is also difficult to exchange data between proprietary systems which code the same types of data differently. Another problem arises from the fact that different medical establishments store some data in digital exchangeable form and other data in less portable forms (paper and film); there is currently no universal agreement as to which data is stored in what form.

In the light of aforementioned discussion there exists a need for an effective medical records management method to assess an individual's health.

### BRIEF SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

Exemplary aspect of the present disclosure comprises of a system and method of preventive health management for assessing an individual's health through consolidation and digitization of medical records.

Another Exemplary aspect of the present disclosure is directed towards to a system and method for scheduled exchange and display of an individual's information.

Another exemplary aspect of the present disclosure comprises of an assessment module for assessing the overall health of an individual.

Another exemplary aspect of the present disclosure comprises of an overall wellness score for a complete assessment of overall health of an individual.

Another exemplary aspect of the present disclosure comprises of an interactive displaying of health records enables an individual to self-learn and self-understand health profile.

Another exemplary embodiment of the present disclosure comprises of a timeline module for having a record of the date and timing of the tests conducted.

Another exemplary embodiment of the present disclosure comprises of a module for generating reminders based on the input of information for reminding an upcoming immunization, pending health check-up and vaccination time.

Yet another exemplary embodiment of the present disclosure comprises of a module for assessment of the overall health of the employees in an organisation based on the parameters obtained.

### BRIEF DESCRIPTION OF DRAWINGS

Other objects and advantages of the present invention will become apparent to those skilled in the art upon reading the following detailed description of the preferred embodiments, in conjunction with the accompanying drawings, wherein like reference numerals have been used to designate like elements, and wherein:

FIG. 1A is a diagram depicting a system of preventive health management for assessing an individual's health through consolidation and digitization of medical records, according to an exemplary embodiment of the present disclosure.

FIG. 1B is a diagram depicting modules of the system 102, according to an exemplary embodiment of the present disclosure.

FIG. 2 is a flow chart representing a method of preventive health management for assessing an individual's health through consolidation and digitization of medical records, according to an exemplary embodiment of the present disclosure.

FIG. 3 is a flow chart representing a method of the assessment module, according to an exemplary embodiment of the present disclosure.

FIG. 4A-4B represents an overview of consolidated and digitized medical records, according to an exemplary embodiment of the present disclosure.

FIG. 5 is a representation of medical records associated with body assessment module, according to an exemplary embodiment of the present disclosure.

FIG. 6 is a representation of medical records associated with dental assessment module, according to an exemplary embodiment of the present disclosure.

FIG. 7 is a representation of medical records associated with vision assessment module, according to an exemplary embodiment of the present disclosure.

FIG. 8 is a representation of medical records with a timeline module, according to an exemplary embodiment of the present disclosure.

FIG. 9 is a representation of medical records associated with a document module, according to an exemplary embodiment of the present disclosure.

FIG. 10 is a representation of pricing list generated by an offers module in response to consolidation and digitization of medical records, according to an exemplary embodiment of the present disclosure.

FIG. 11 is a representation of rewards gained by the individual in response to consolidation and digitization of medical records, according to an exemplary embodiment of the present disclosure.

FIG. 12 is a representation of basic profile of the individual, according to an exemplary embodiment of the present disclosure.

FIG. 13 is a representation of family history of a individual, according to an exemplary embodiment of the present disclosure.

FIG. 14 is a representation of allergies, according to an exemplary embodiment of the present disclosure.

FIG. 15 is a representation of immunizations, according to an exemplary embodiment of the present disclosure.

FIG. 16 is a representation of medications, according to an exemplary embodiment of the present disclosure.

FIG. 17 is representation of an overall wellness score obtained in an organization along with a graphical representation of the age of the employees' vs the number of employees in an organization, according to an exemplary embodiment of the present disclosure.

FIG. 18 is a representation of medical conditions of the working employees, according to an exemplary embodiment of the present disclosure.

FIG. 19 is a representation of lifestyle factors of the frequency of a lifestyle related habit vs the total number of employees taken for study, according to an exemplary embodiment of the present disclosure.

FIG. 20 is a representation of a landing page of the appointment engine module, according to an exemplary embodiment of the present disclosure.

FIG. 21 is a representation of an extension of the offers provided for scheduling an appointment, according to an exemplary embodiment of the present disclosure.

FIG. 22 is a representation of a choice of the diagnostic center recommended as per the requirement of the individual, according to an exemplary embodiment of the present disclosure.

FIG. 23 is a representation of the payment procedure to be adapted for finalizing an appointment, according to an exemplary embodiment of the present disclosure.

### DETAILED DESCRIPTION

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another.

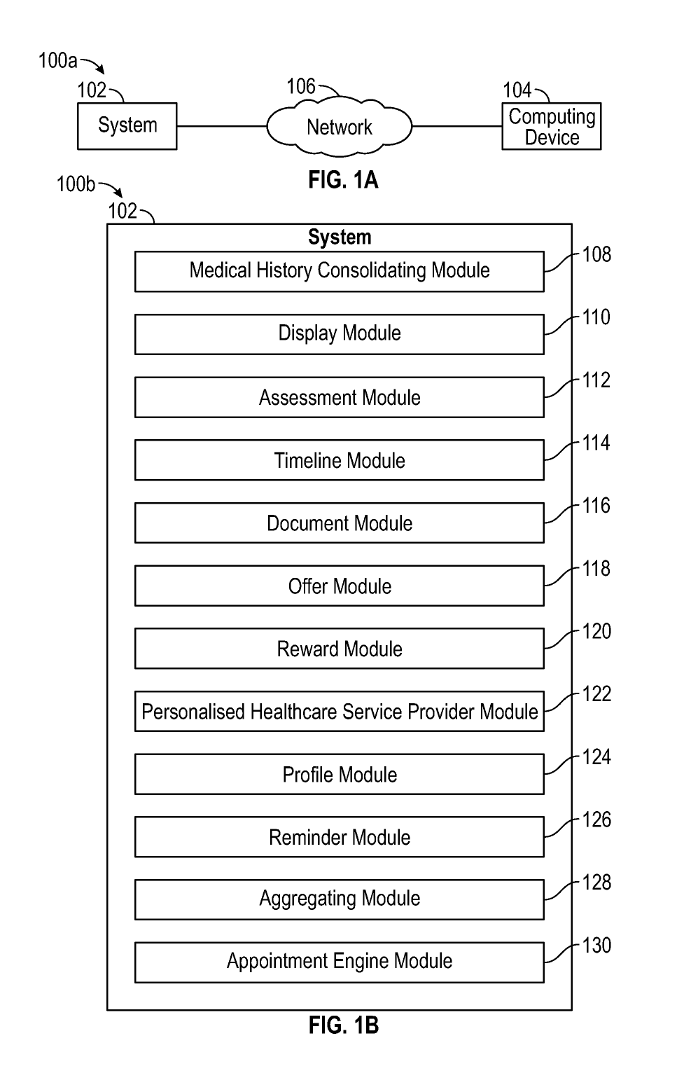

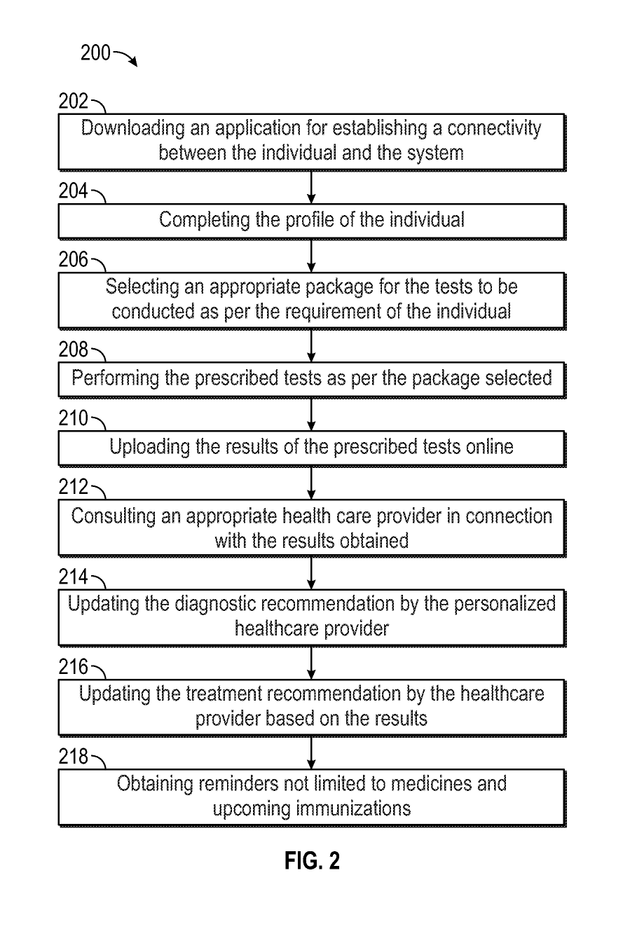

Referring to FIG. 1A, is a diagram 100a depicting a system of preventive health management for assessing an individual's health through consolidation and digitization of medical records, according to an exemplary embodiment of the present disclosure. The present disclosure may be implemented in a computing device 104 and a system 102 connected over a network 106. The network 106 may include, but not limited to, an Ethernet, a local area network (LAN), or a wide area network (WAN), e.g., the Internet, or a combination of networks. The present system 102 may be accessed as a web base application and/or a mobile application.

The system 102 is configured to consolidate the medical history of individuals wherein the individuals have to take a picture of the records, prescriptions and readings from medical devices etc. to consolidate the information into a single cloud repository, which then can be accessed by them, their family and service providers from anywhere and at any time in a digital format. Here the system 102 may be referred to as a software application, dedicated medical software application, mobile application and the like without limiting the scope of the disclosure. The computing device 104 is a individual device configured to provide inputs to the system 102. Here the computing device 104 may include a computer, laptop, mobile, and the like without limiting the scope of the disclosure. The individual may be allowed to take the picture of the health results provided as a report and upload to the system 102 through the device 104.

Referring to FIG. 1B is a block diagram 100b, depicting modules involved in the system 102, according to an exemplary embodiment of the present disclosure. The system 102 includes a medical history consolidating module 108, a display module 110, an assessment module 112, a timeline module 114, a document module 116, offers module 118, a reward module 120, a personalised healthcare service provider module 122, a profile module 124, a reminder module 126, an aggregating module 128 and an appointment engine module 130.

The medical history consolidating module 108 is configured to consolidate the medical history of individuals. Here the medical history records may include prescriptions, readings from medical devices and the like without limiting the scope of the disclosure. The display module 110 is configured to display the basic details of an individual as in a profile, medical reports, vital statistics and the like in various colour shades to enable the individual to see one's parameter readings displayed from previous tests conducted. The display module 110 configured for interactively displaying a consolidated medical records of the individual in response to analyzing the medical records, whereby the interactive displaying of health records enables an individual to self-learn and self-understand health profile; the assessment module 112 is configured to analyse the details obtained by various parameters subjected to tests. The module is further configured to assess one's parameters as in height, weight, blood glucose levels, blood pressure and the like without limiting the scope of the disclosure. The module is further enabled to assess parameters involving specific areas as in vision assessment, dental assessment and the like. The module enables to assess the risk factor involved based on the obtained health parameters. The timeline module 114 is configured to provide the individual with the timeline of health checks in order to keep a track of the medical records. The document module 116 enables the individual to have an aggregate of the medical records and other documents obtained from various sources. The sources may not be limited to capturing a picture of the records through a camera enabled hand device, uploading of a document in one off online or offline methods. The documents may include but not limited to medical records, diagnostic images, and payment vouchers. The offers module 118 enables the individual to know about any special offers or discounts offered for the tests prescribed. The reward module 120 enables the individual to obtain rewards on completion of prescribed processes.

The personalised access provider module 122 is configured to provide the personalized access to healthcare service providers based on the chronic condition of the individual; the profile module 124 enables the profile of the individual and the family members along with the medications and immunization programmes to be updated and the reminder module 126 enables an individual to be reminded regarding any upcoming immunization requirement, medications, medical checkup and the like; the aggregating module 128 provides an aggregate of the medical history at an organizational level for the corporate employees. The module enables to assess the overall wellness score of the organisation based on the assessments of various medical conditions, lifestyle factors, age group and the like; and the appointment engine module is basically a self-advisory module wherein the individual is enabled to perform a self-check in service based on the requirements of health checks.

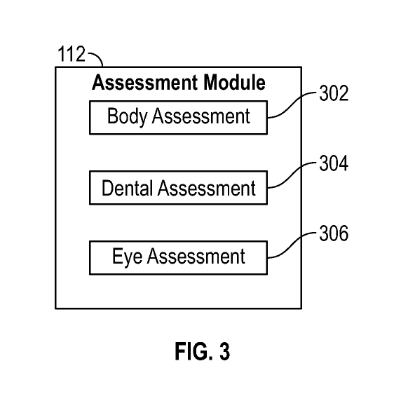

Referring to FIG. 2 is a flow diagram 200, representing a method of preventive health management for assessing an individual's health through consolidation and digitization of medical records, according to an exemplary embodiment of the present disclosure. The method starts at step 202 by downloading an application; further a profile of the individual may be completed at step 204; an appropriate package may be selected as per the requirement at step 206; further the prescribed tests are performed at step 208; a results of the tests obtained may be uploaded online at step 210; a visit may be paid to an appropriate health care provider in connection with the results obtained at step 212; a diagnostic recommendation by the health care provider may be updated as in step 214; an uploading of the treatment details as suggested by the health care provider can be done at step 216; updating the information related to medicines, immunizations for obtaining adequate reminders can be done at step 218.

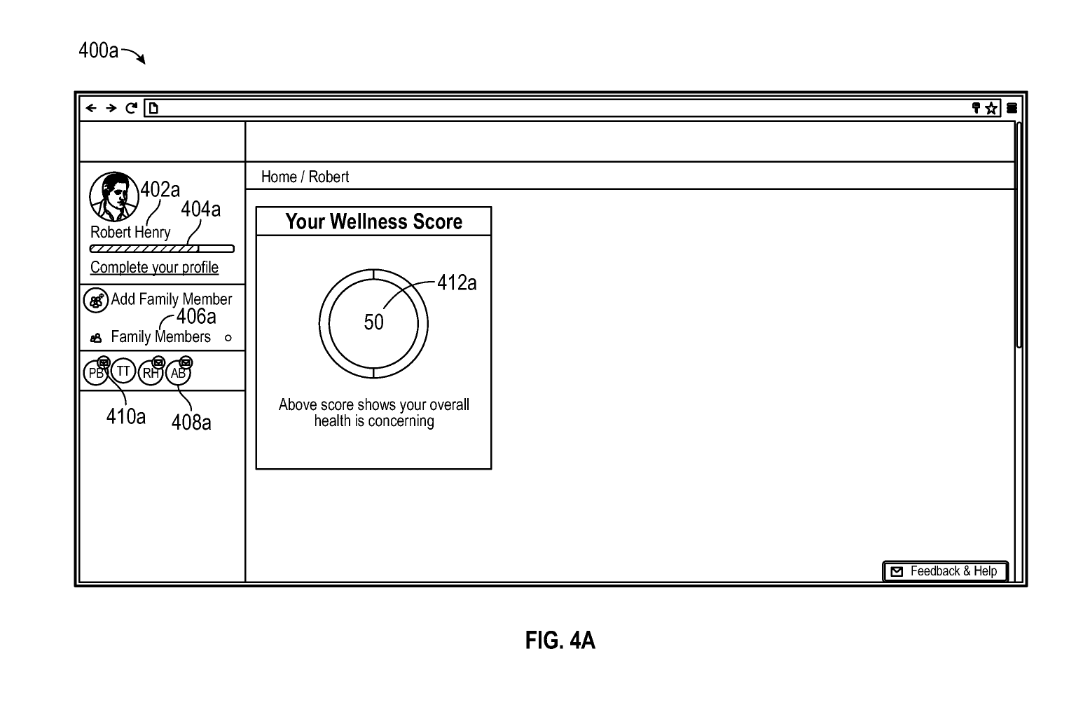

Referring to FIG. 3 is a flow diagram 300, representing a method of assessment module 112, according to an exemplary embodiment of the present disclosure. The module starts with a body assessment module 302 with regard to the score of a parameter obtained. Further, a dental assessment module 304 with regards to a dental report obtained is done and finally a vision assessment module 306 with regards to eye reports obtained.

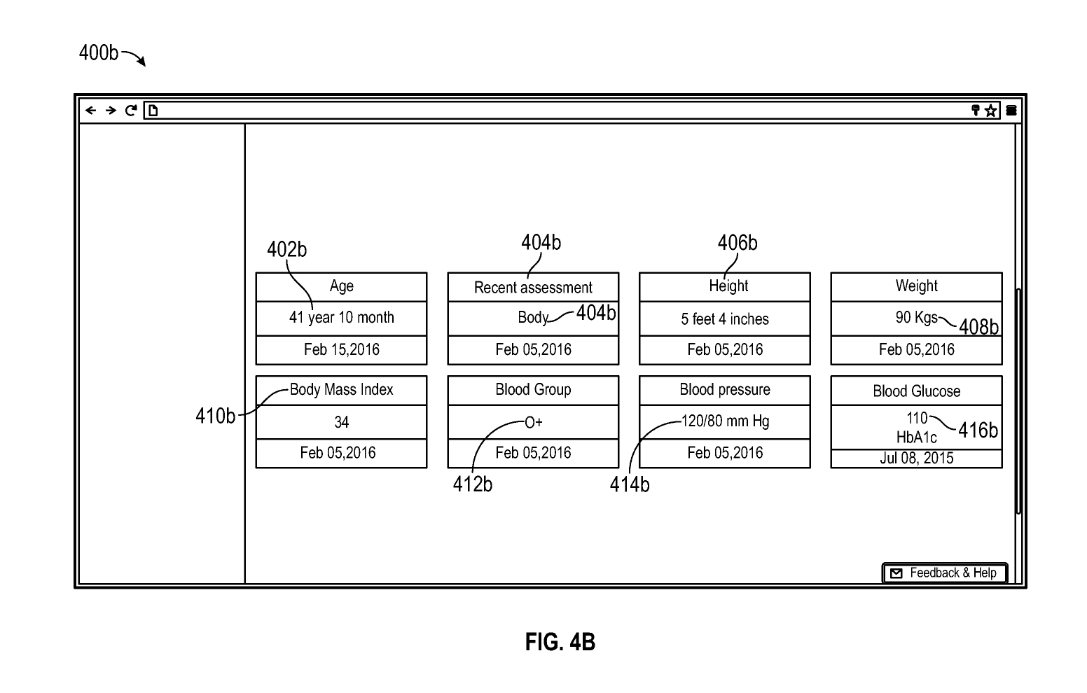

Referring to FIG. 4A is a diagrammatic representation 400a, of an overview of preventive health management for assessing an individual's health through consolidation and digitization of medical records comprising of consolidation and display of an individual's basic details as in name of the individual 402a and completion of the details of the profile as a shaded bar 404a. Further the consolidation and display of individual's family member's details 406a comprising of an initial alphabet of the name of the family members 408a along with a mail logo 410a which would enable the individual to know about any pending mails from the corresponding member of the family. Further, an overall wellness score 412a is displayed which would enable the individual to have an idea about one's general health.

Referring to FIG. 4B is a diagrammatic representation 400b, of an overview of preventive health management for assessing an individual's health through consolidation and digitization of medical records comprising of further details of the individual for display comprising of details of an individual's age in years and months on a given date 402b, a recent assessment of one of the parameters 404b, a height of the individual 406b, a weight of the individual 408b, a body mass index 410b, a blood group 412b, a blood pressure 414b, a blood glucose levels 416b which may be fasting blood glucose levels, post prandial blood glucose levels, HbA1c levels and the like according to an exemplary embodiment of the present disclosure.

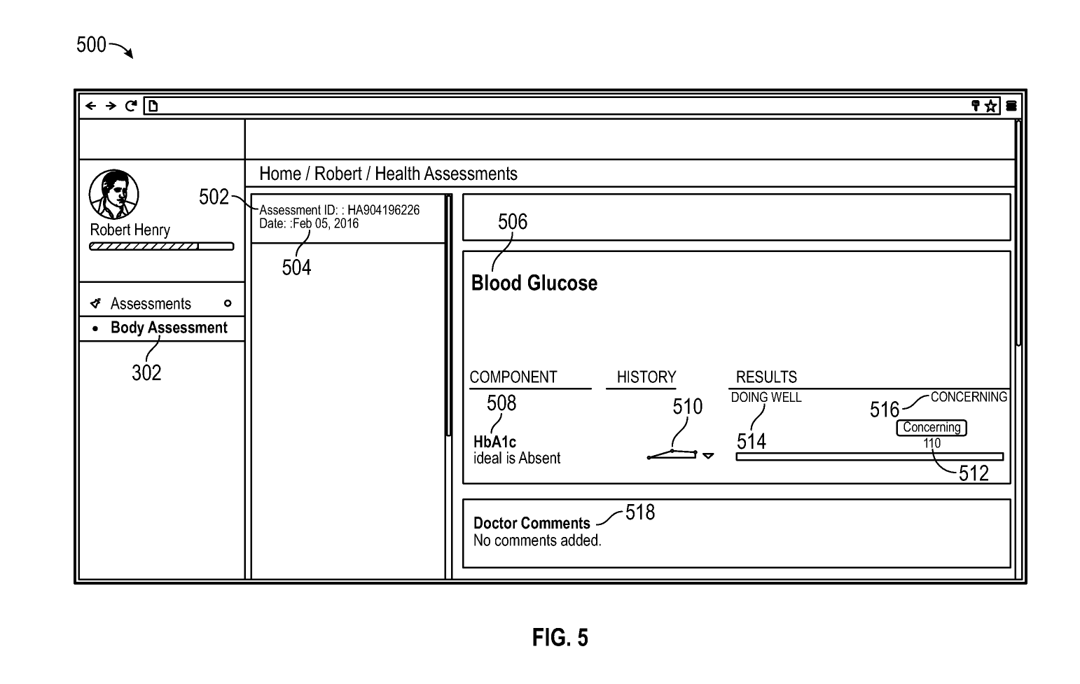

Referring to FIG. 5 is a representation of medical records associated with body assessments module 302, according to an exemplary embodiment of the present disclosure. The disclosure comprises of a drop down menu which includes an assessment ID 502 allocated to the individual along with a date of allocation 504. This when clicked upon further enables the display of a parameter as in blood glucose 506 and the like. Further the component of the parameter assessed as in HbA1c levels in blood glucose 508 is also displayed. A graphical representation 510 of a history of the levels of the components tested is displayed along with the value obtained 512. The assessments further comprising of tags as in doing well 514 and concerning 516 which is enabled to be displayed in various colour shades to enable the individual to assess based on one's parameter reading from previous tests conducted. The disclosure may be applicable to cases like hypertension wherein systolic and diastolic blood pressure is also represented in a graphical format. A health care provider's analysis in the form of a comment may be provided at 518.

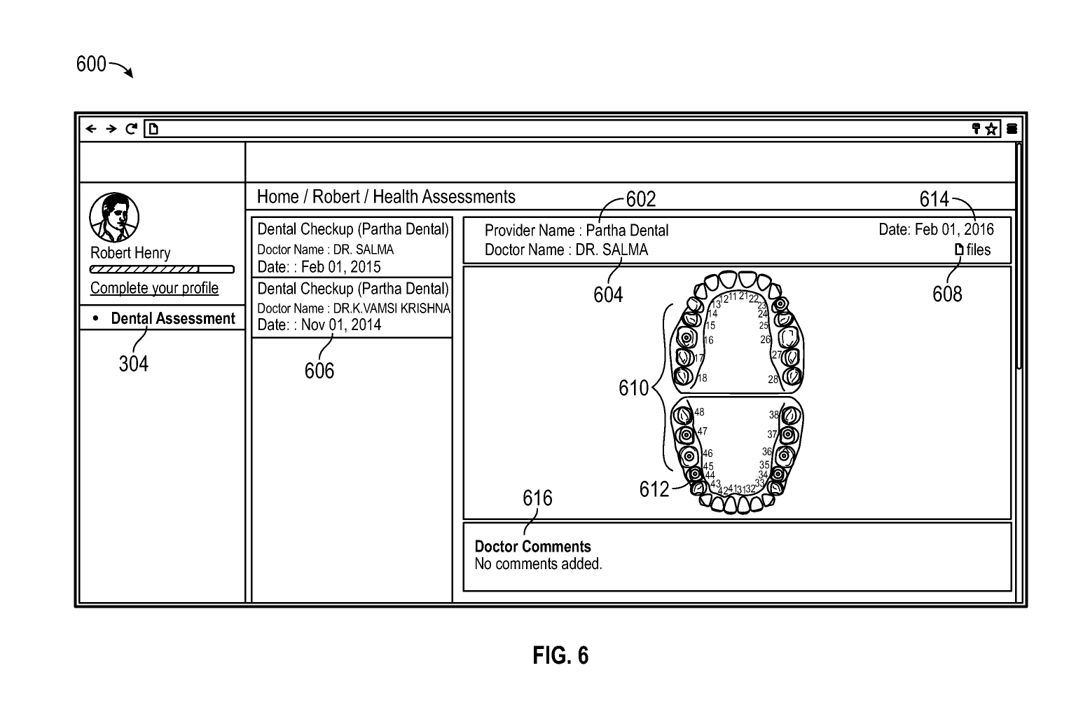

Referring to FIG. 6 is a representation 600, of medical records associated with dental assessment module 304, according to an exemplary embodiment of the present disclosure. The disclosure comprises of the details as in a name of the diagnostic center 602, a name of the dentist 604, a date on which a previous checkup was conducted 606 and an uploaded document of the dental report 608. Further a diagrammatic representation 610 of a dental layout is displayed which may differ as per the age of the individual wherein the display may change for example: for kids only 28 teeth may be displayed, for old aged people the extracted tooth need not be displayed, for adults a complete set may be displayed and the like without limiting the scope of the disclosure. Crowns, specific molar, premolars, canines and incisors and a plurality of teeth in need of treatment are displayed with a dot as in 612. Date of current checkup 614 is displayed. A diagnosis in the form of the dentist's comment 616 which has been concluded post the checkup is displayed as a part of personalized healthcare service provider module 122.

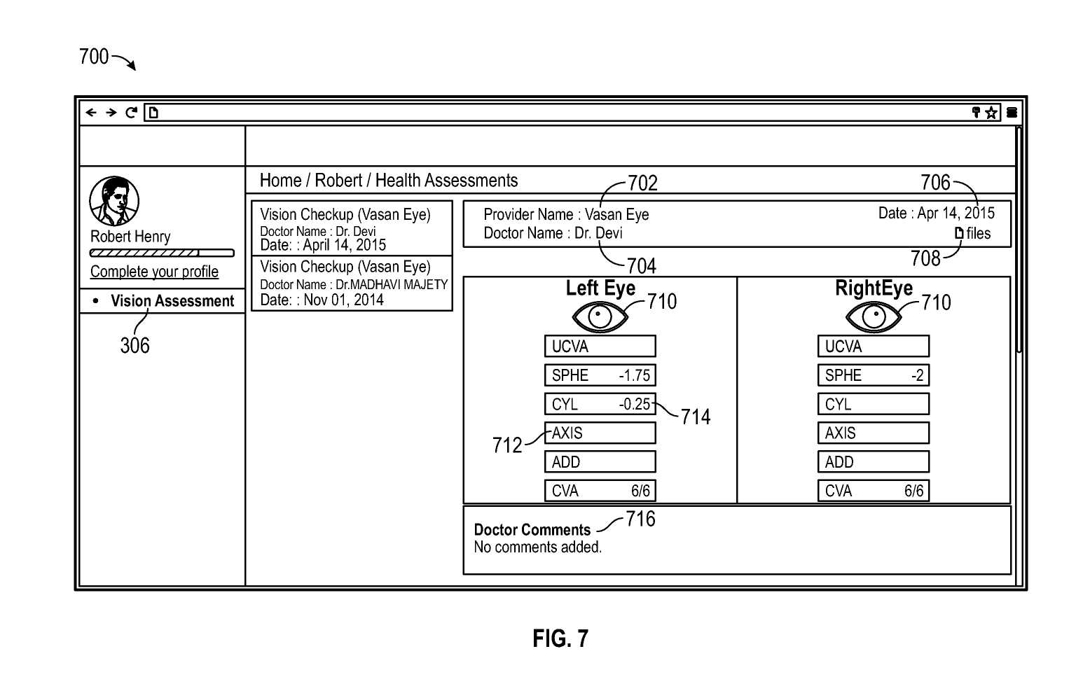

Referring to FIG. 7 is a representation 700, of medical records associated with vision assessment module 306, according to an exemplary embodiment of the present disclosure comprising of the details as in the name of the diagnostic center 702, name of the ophthalmologist consulted 704, a date on which a checkup was conducted 706 and an uploaded document of the eye report 708. Further a diagrammatic representation 710 of both the left eye and the right eye is displayed. A name of each of the parameters related to an eye 712 accompanied by a score 714 is displayed. The disclosure finally includes a provision for an opinion by the ophthalmologist 716 as a case by case basis depending upon the requirement as a part of personalized healthcare service provider module 122.

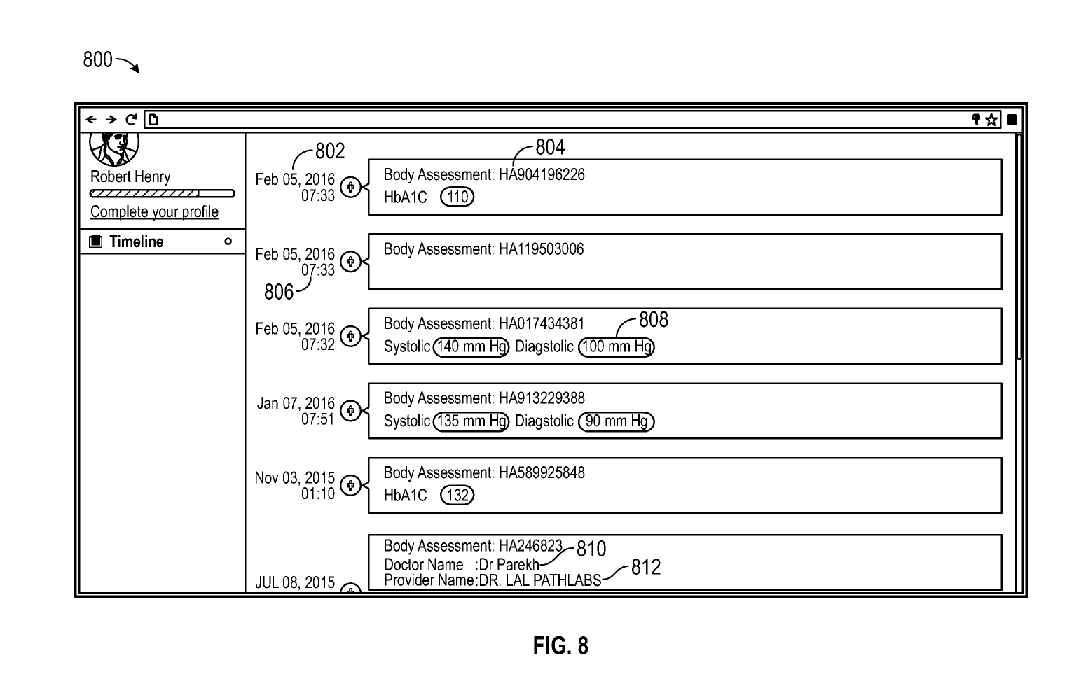

Referring to FIG. 8 is a representation 800, of medical records with a timeline module, according to an exemplary embodiment of the present disclosure comprising of details of series of checkups displayed along with a date 802, an assessment ID 804, a time during which the parameter was measured 806 and a reading of the parameter 808. A health care provider's name 810 and a diagnostic center 812 conducting the test are mentioned in the disclosure.

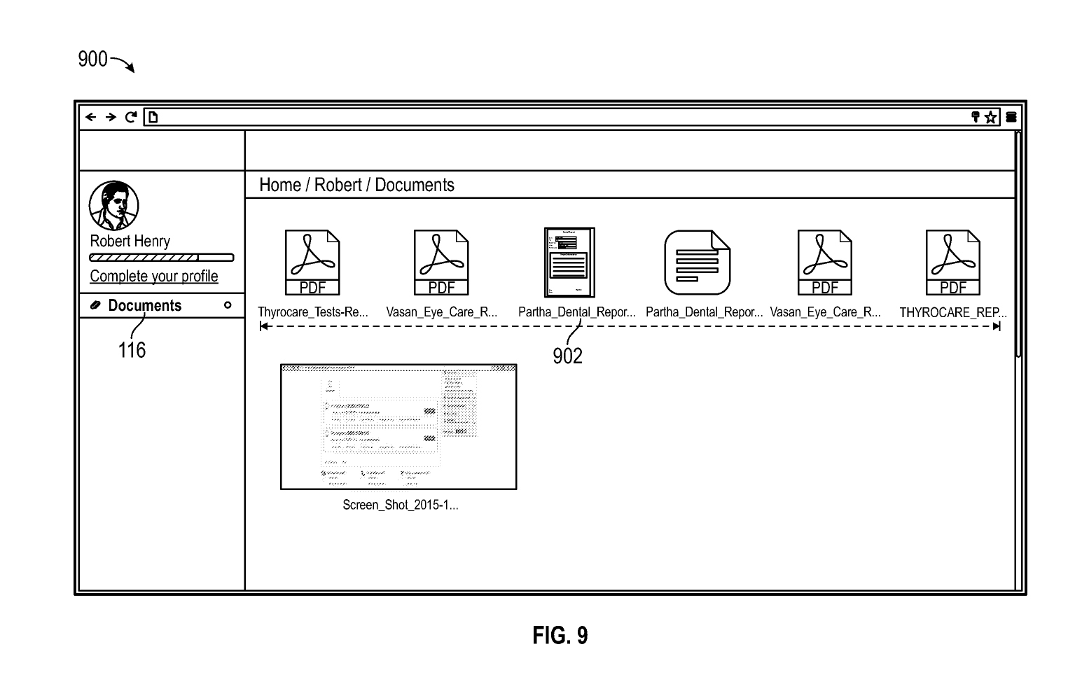

Referring to FIG. 9 is a diagrammatic representation 900, medical records associated with a document module 116, according to an exemplary embodiment of the present disclosure comprising of a consolidated space for uploading and storing medical records and other relevant documents obtained from various sources. Medical records 902 may include but not limited to blood glucose reports, dental assessment reports, and vision assessment reports. Other documents may include but not limited to diagnostic images and payment vouchers.

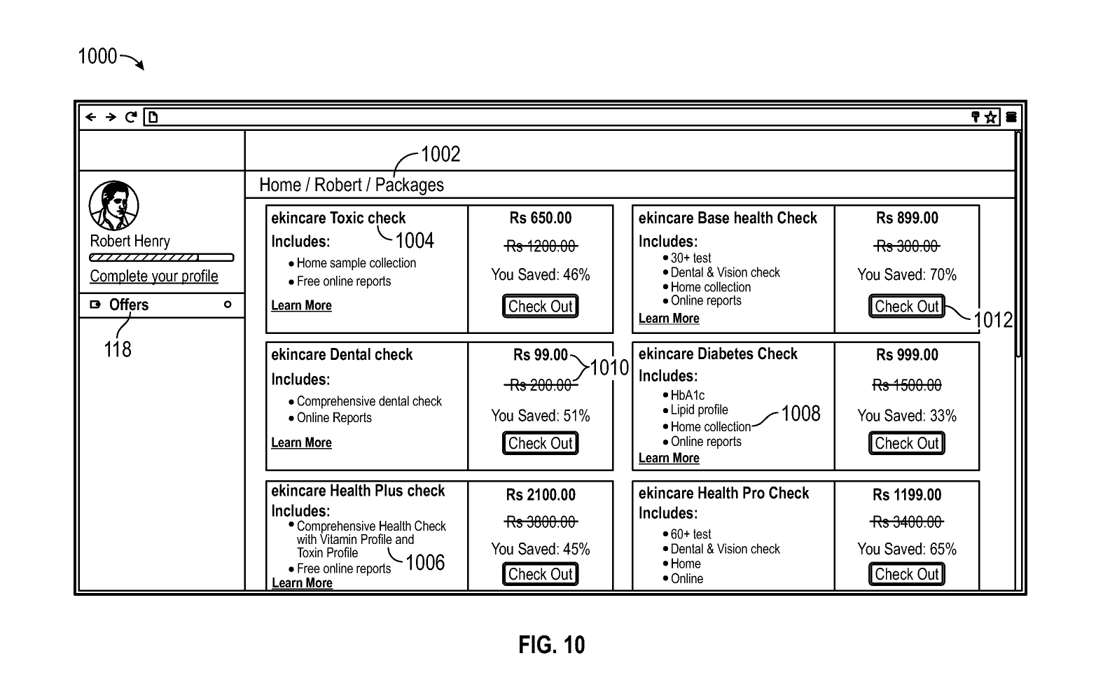

Referring to FIG. 10 is a representation 1000, of pricing list generated by an offers module 118 in response to consolidation and digitization of medical records, according to an exemplary embodiment of the present disclosure comprising of complete details of a package offered 1002 which further include a name of the checkup desired or recommended 1004, names of the tests 1006 included in the prescribed package not limited to vitamin checks, comprehensive dental checks, vision checks, lipid profiles and blood glucose tests. A package offered 1002 inclusive of free online upload of the reports, home collection of blood samples 1008 and the like without limiting the scope of the disclosure. Any discounts offered along with slashed prices 1010 are also displayed. A check out option 1012 is provided to continue with further payment related formalities.

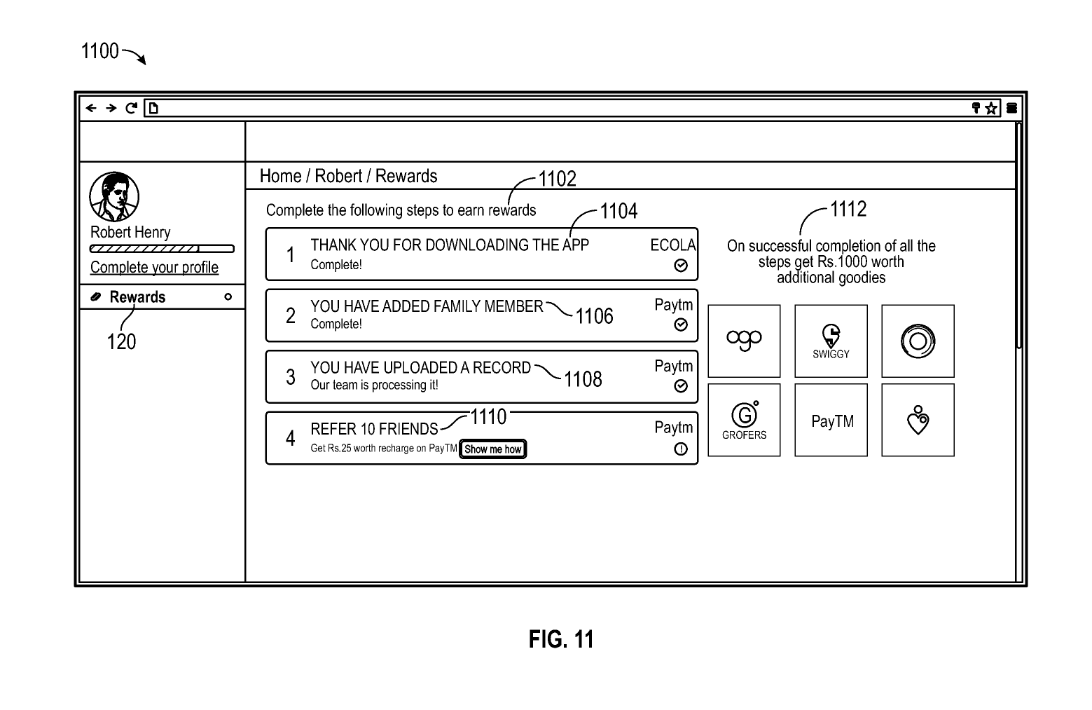

Referring to FIG. 11 is a representation 1100, of rewards gained by the individual in response to consolidation and digitization of medical records, according to an exemplary embodiment of the present disclosure comprising of step by step instructions required to be completed 1102 which would enable the individual to obtain a reward. The first step 1104 includes a thanks message for having downloaded the application. A second step 1106 includes a provision to add a family member. A third step 1108 includes a provision to upload a medical record and, a fourth step 1110 includes a provision to refer an application not limited to a certain number of friends. This enables the individual to obtain an offer in a cash based service 1112. This may be referred to as the reward module 120.

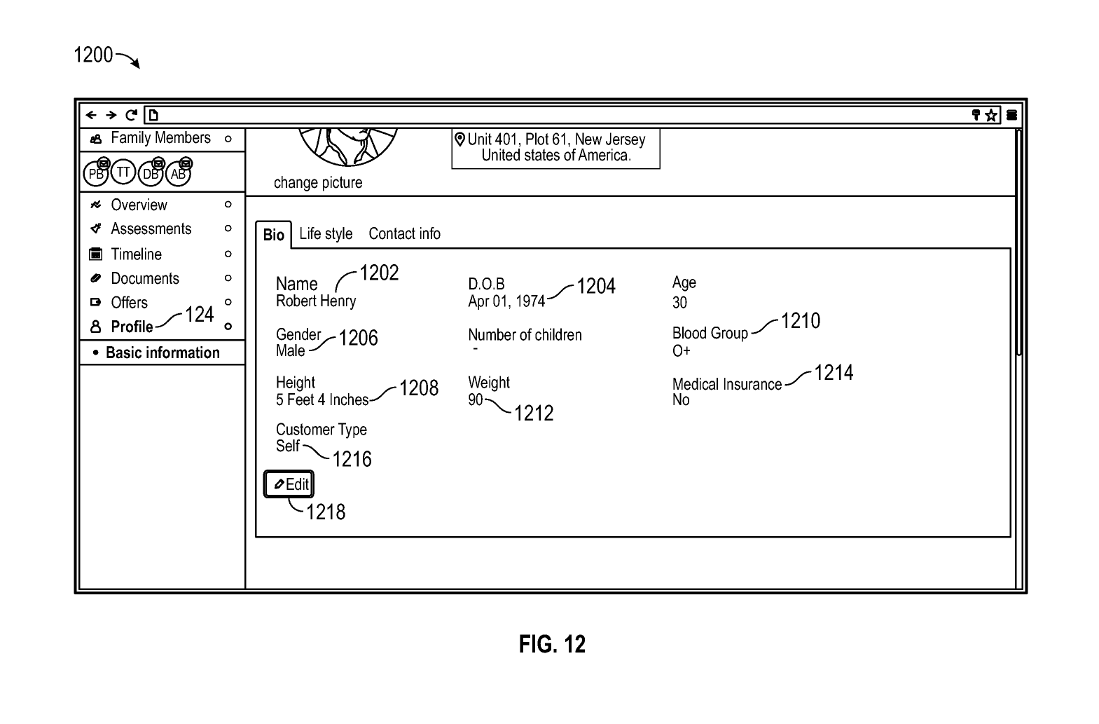

Referring to FIG. 12 is a representation 1200 of basic profile of the individual, according to an exemplary embodiment of the present disclosure comprising of a name of the individual 1202, date of birth of the individual 1204, gender of the individual 1206, height of the individual 1208, blood group of the individual 1210, weight of the individual 1212, medical insurance if at all held by the individual 1214 and a customer type option 1216 wherein the individual may provide the detail of whether it is one's own account or that of a family member. An edit option 1218 enables the individual edit the details mentioned as per the requirement. This may be referred to as the profile module 124.

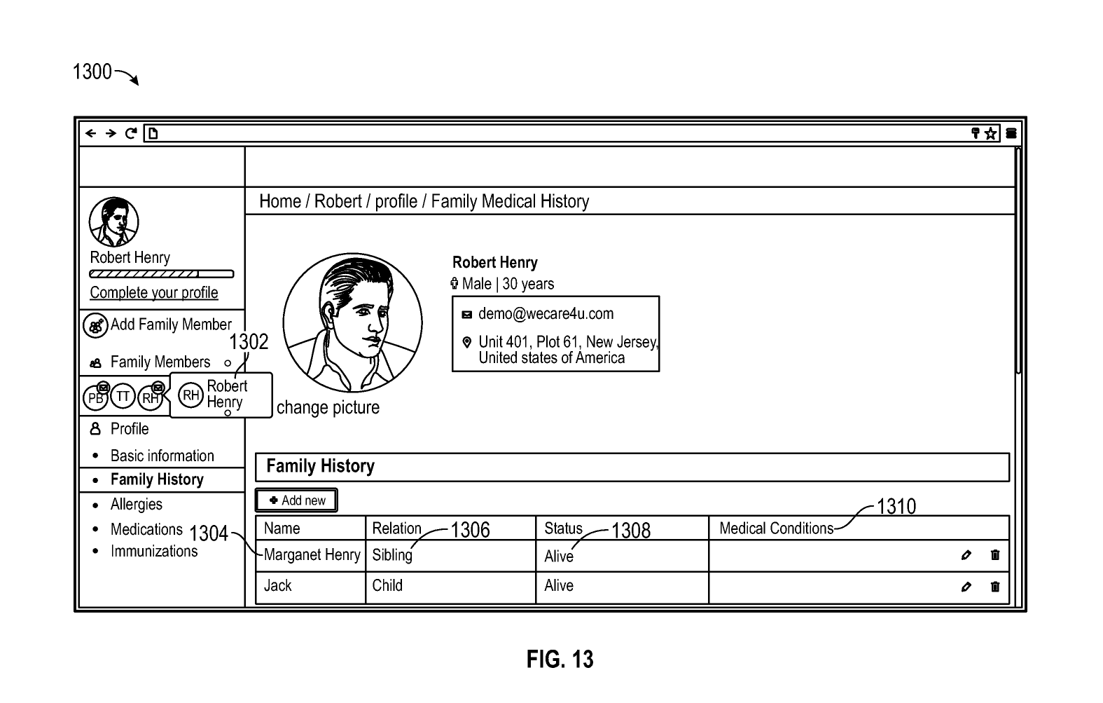

Referring to FIG. 13 is a representation 1300 of family history of a individual, according to an exemplary embodiment of the present disclosure comprising of a detail about the family member wherein their initials are depicted in capitals 1302. Further a tabular input is displayed wherein the individual may include but not limited to details as name of the family member 1304, relation with the individual 1306, status of the family member 1308 as in alive or dead and any medical condition 1310 associated with the family member in question.

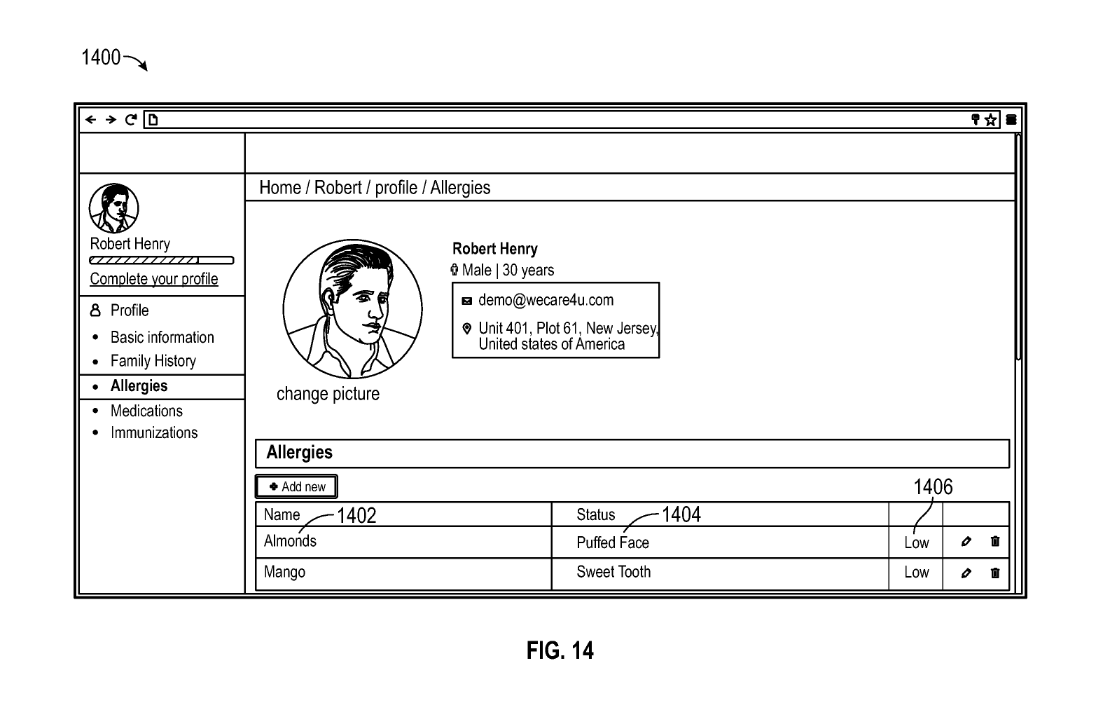

Referring to FIG. 14 is a representation of allergies 1400, according to an exemplary embodiment of the present disclosure comprising of a provision to name an ingredient or food 1402 which has led to an allergic reaction in the individual. A kind of reaction as in puffed face, rashes, sweet tooth and the like 1404 may also be mentioned. Further at 1406 the intensity of the reaction whether low or high as the case may be mentioned.

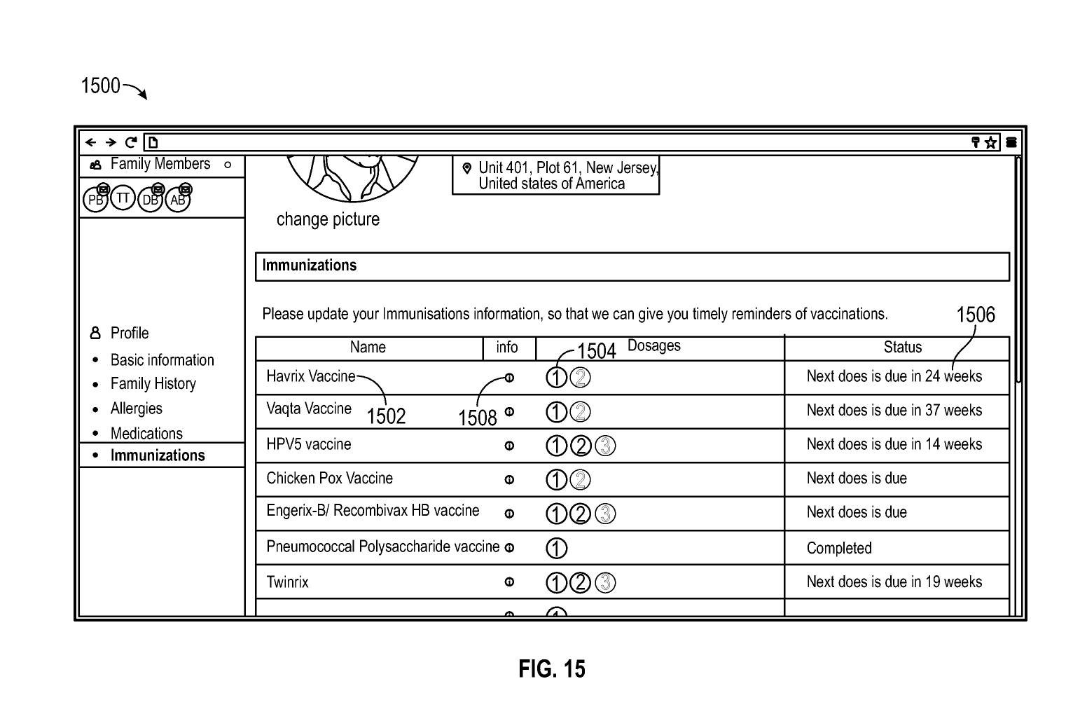

Referring to FIG. 15 is a representation of immunizations 1500, according to an exemplary embodiment of the present disclosure comprising. A name of a vaccination 1502 may be mentioned followed by a number indicating the dosage already taken 1504 and a status option 1506 would enable the individual to know about the due date for the next dosage. An info option 1508 enables the individual to have information about the vaccine of updating information by the individual wherein a reminder can be sent to the individual regarding any upcoming immunization requirement as a part of the reminder module 126.

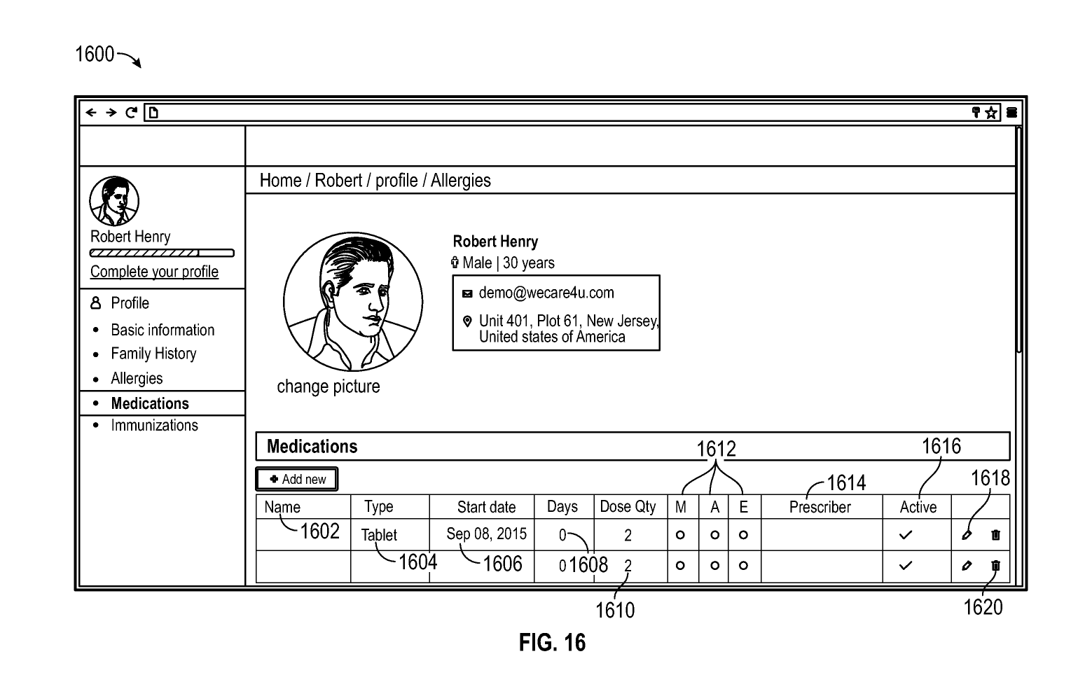

Referring to FIG. 16 is a representation of medications 1600, according to an exemplary embodiment of the present disclosure, comprising of an option wherein the name of the medicine being used is named 1602, a dosage formulation as in tablet, syrup, capsule and the like is mentioned at 1604, a date of starting the medication 1606, number of days when the medication was taken 1608, number of dosages 1610, a time period for the medicine as in morning, afternoon or evening 1612, a prescriber of the medicine 1614 and is the medication active 1616? An editing option 1618 and a delete option 1620 to delete the options may be provided once the medication dose is over or discontinued as the case may be.

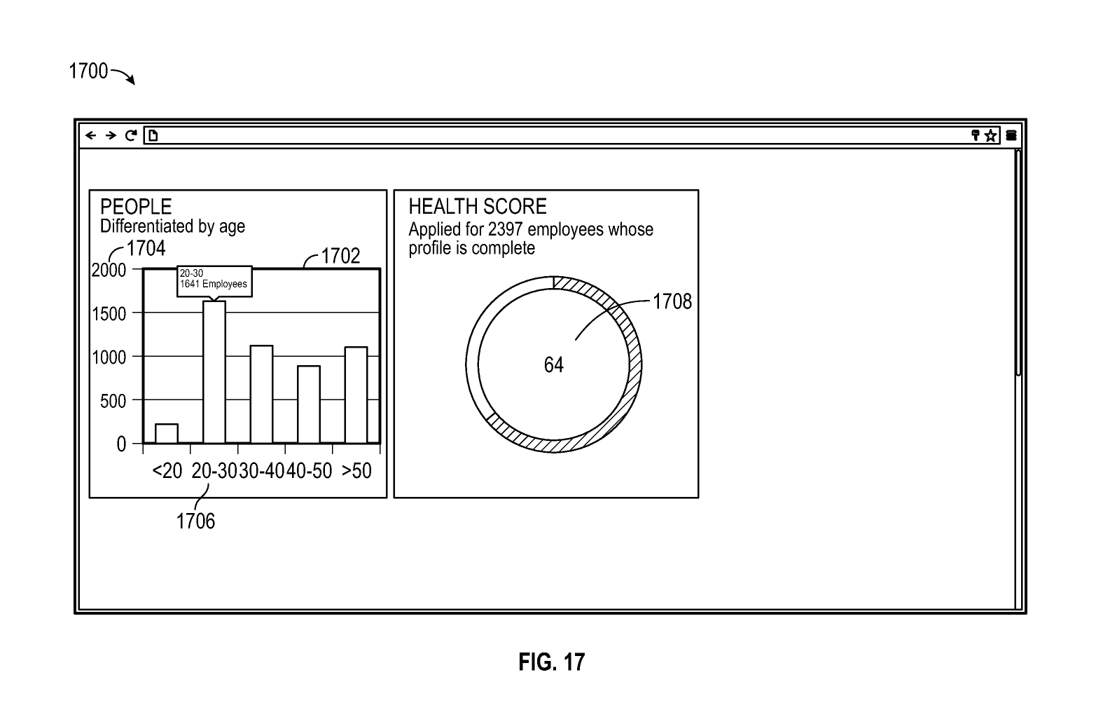

Referring to FIG. 17 is representation 1700 of an overall wellness score obtained in an organization along with a graphical representation of the age of the employees' vs the number of employees in an organization, according to an exemplary embodiment of the present disclosure. The graphical representation 1702 is done by considering the number of employees who had undergone the health check-up 1704 vs age of the employees 1706 as in the age group in their <20 yrs, 20-30 yrs, 30-40 yrs, 40-50 yrs, >50 yrs and the like. The overall wellness score 1708 is displayed which is obtained by subjecting the employees through various tests. The overall test result obtained enables the organisation to have an idea about the overall health score of the employees which is in turn the overall wellness score 1708 of the organisation. The module may be referred to as an aggregate module 128 as the aggregation enables an organization and one or more individuals to self-learn and self-understand health profile of one more individuals.

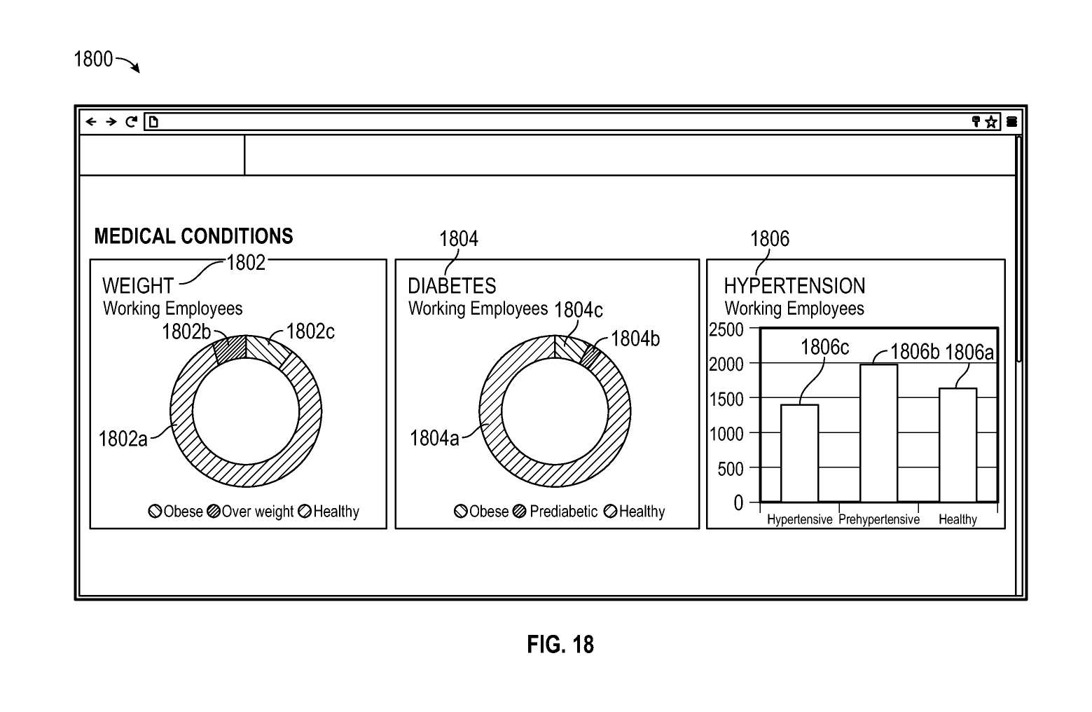

Referring to FIG. 18 is a representation 1800, of medical conditions of the working employees, according to an exemplary embodiment of the present disclosure. The parameters which are assessed include but not limiting to the weight 1802 of the working employees as in healthy 1802a, overweight 1802b and obese 1802c. The second parameter includes an assessment of the diabetes levels 1804 in an employee as in healthy 1804a, pre diabetic 1804b and diabetic 1804c. Graphical representations of hypertension amongst the number of working employees 1806 include an assessment of the levels of hypertension as healthy 1806a, pre hypertensive 1806b and hypertensive 1806c.

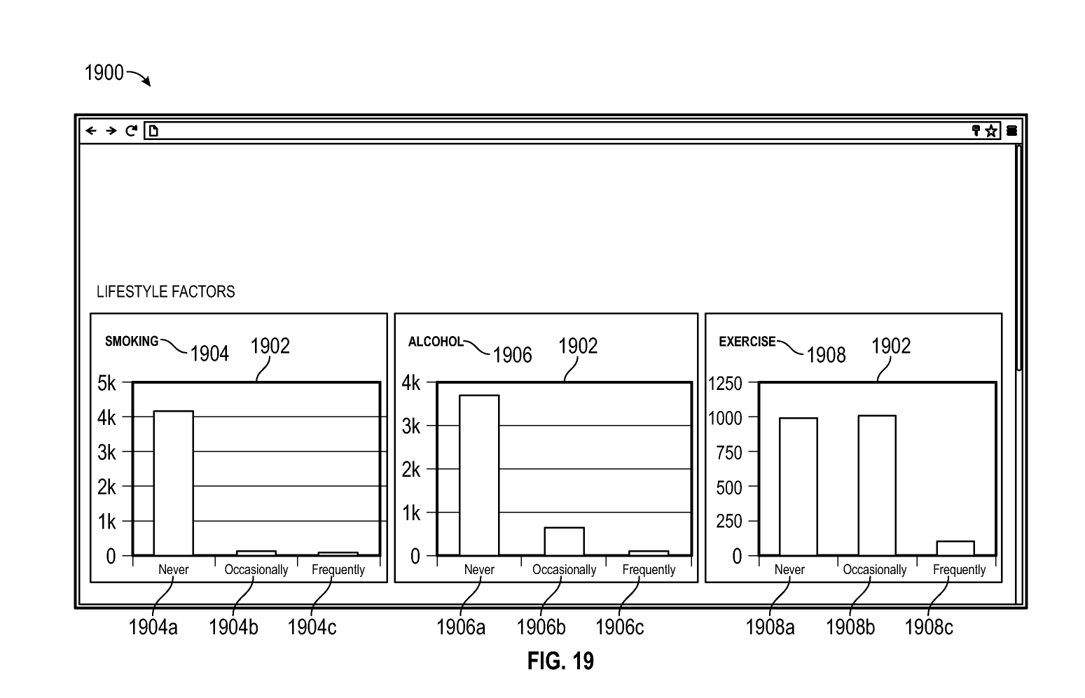

Referring to FIG. 19 is a representation 1900, comprising of lifestyle factors and the frequency of a lifestyle related habit vs the total number of employees taken for study, according to an exemplary embodiment of the present disclosure. The graph 1902 includes comparison by considering various lifestyle habits as in smoking 1904 wherein the total number of employees are assessed based on the frequency of smoking habits as in never 1904a, occasionally 1904b and frequently 1904c. Further the graph 1902 includes the lifestyle habits as in alcohol 1906 wherein the total number of employees are assessed based on the frequency of consumption of alcohol as in never 1906a, occasionally 1906b and frequently 1906c. Further the disclosure also includes the graph representing the exercise habits 1908 as in never 1908a, occasionally 1908b and frequently 1908c.

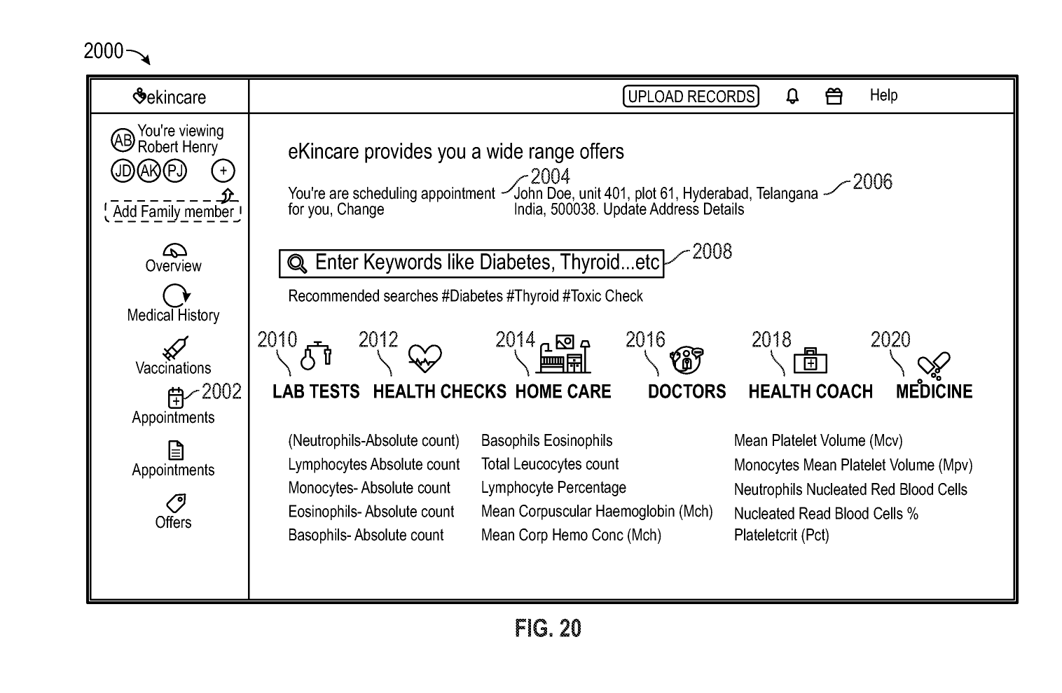

Referring to FIG. 20 is a representation 2000, of a landing page of the appointment engine module, according to an exemplary embodiment of the present disclosure wherein the appointments 2002 comprises of a statement alerting the individual as “you are scheduling an appointment for you” 2004. Appropriate details of the communication address 2006 are provided. A search engine 2008 is provided which enables the individual to type requisite keyword/keywords which may be related to the requisite health checkup into consideration for example; diabetes, thyroid, toxic check and the like without limiting the scope of the disclosure. Further various options are provided as per the requirement of the individual which may not be limited to lab tests 2010, Heath checks 2012, Home care facilities 2014, Doctors 2016, Health coach 2018 and medicines 2020.

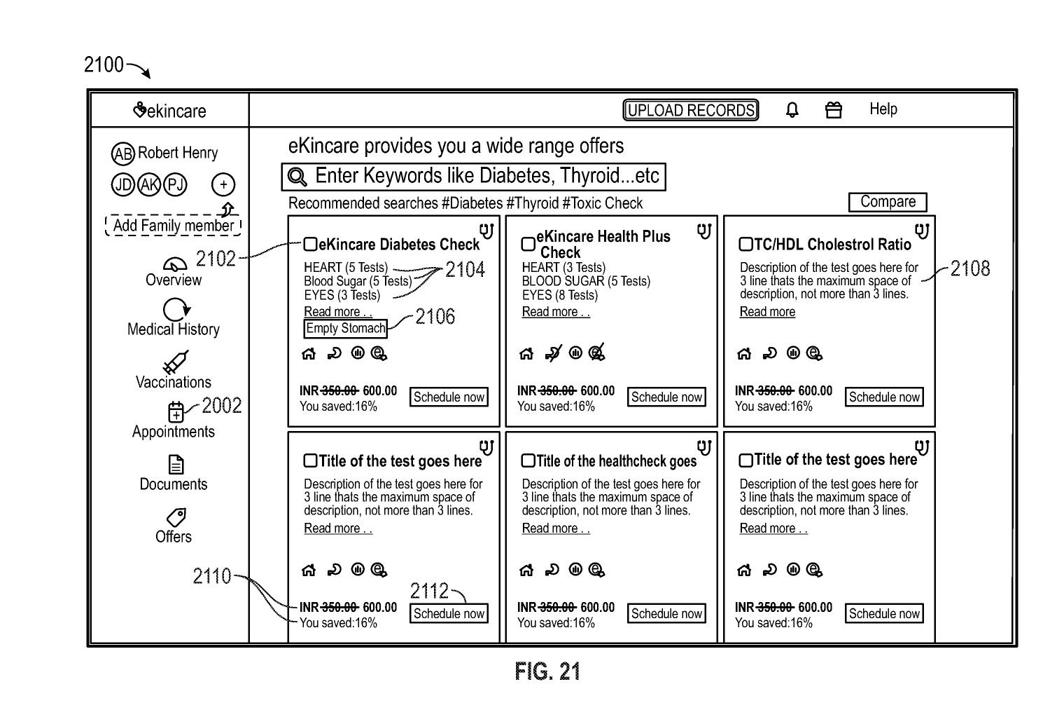

Referring to FIG. 21 is a representation 2100, of an extension of the offers provided for scheduling an appointment, according to an exemplary embodiment of the present disclosure. The figure forms a part of the appointments engine module 2002. A title of the test to be conducted 2102 may be displayed. In the particular title of the test 2102 there are further sub tests 2104 which are needed to be conducted. For example; 5 tests for detecting heart ailments, 5 tests for blood sugar, 3 tests for eye and the like without limiting the scope of the disclosure. The pre requisite conditions like empty stomach 2106 and the like for conducting the tests is mentioned so that the individual comes prepared accordingly for the tests. The total price of the tests chosen to be performed along with any applicable discount 2110 is also displayed. A schedule now 2112 button enables the individual to schedule the appointment as per the requirement.

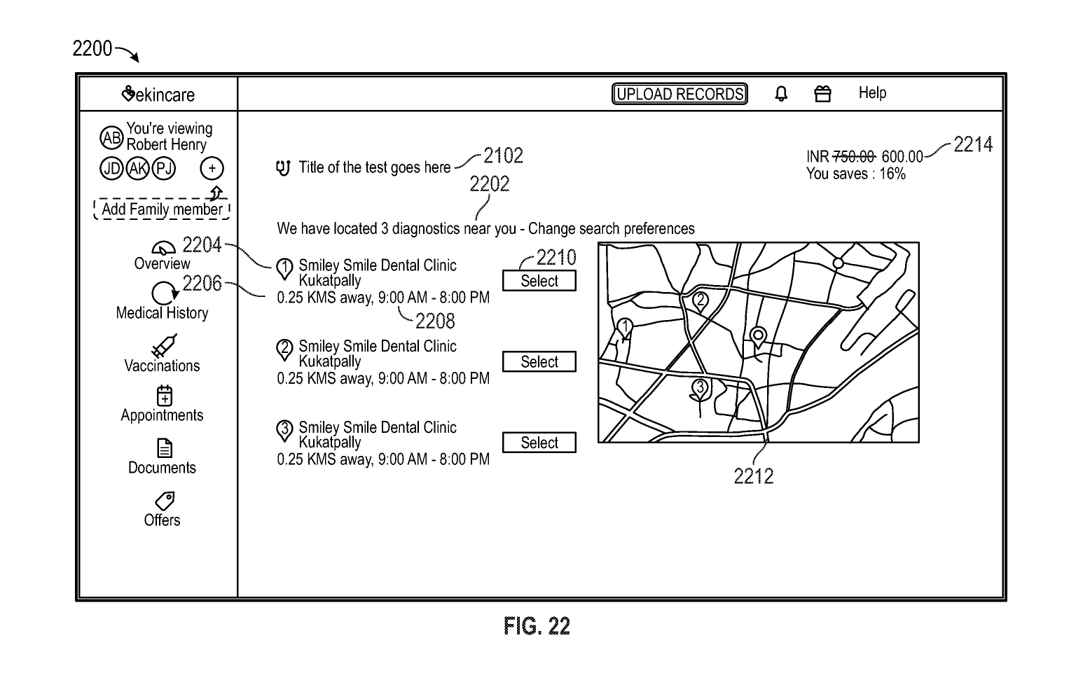

Referring to FIG. 22 is a representation 2200, of a choice of the diagnostic center recommended as per the requirement of the individual, according to an exemplary embodiment of the present disclosure. The figure forms a part of the appointments engine module 2002. The individual is enabled to feed the title of the test 2102 recommended. This further enables the system for location of diagnostic centers nearer to the individual's location 2202. Further the name of the diagnostic center 2204 is enabled to be displayed along with the distance 2206 from an individual's current location. A time 2208 representing the working hours of the diagnostic center is displayed. A diagrammatic representation of an area 2212 with the locations of similar diagnostic centres around an individual's occupancy area is displayed. The fee applicable for the tests recommended along with the discount 2214 is displayed.

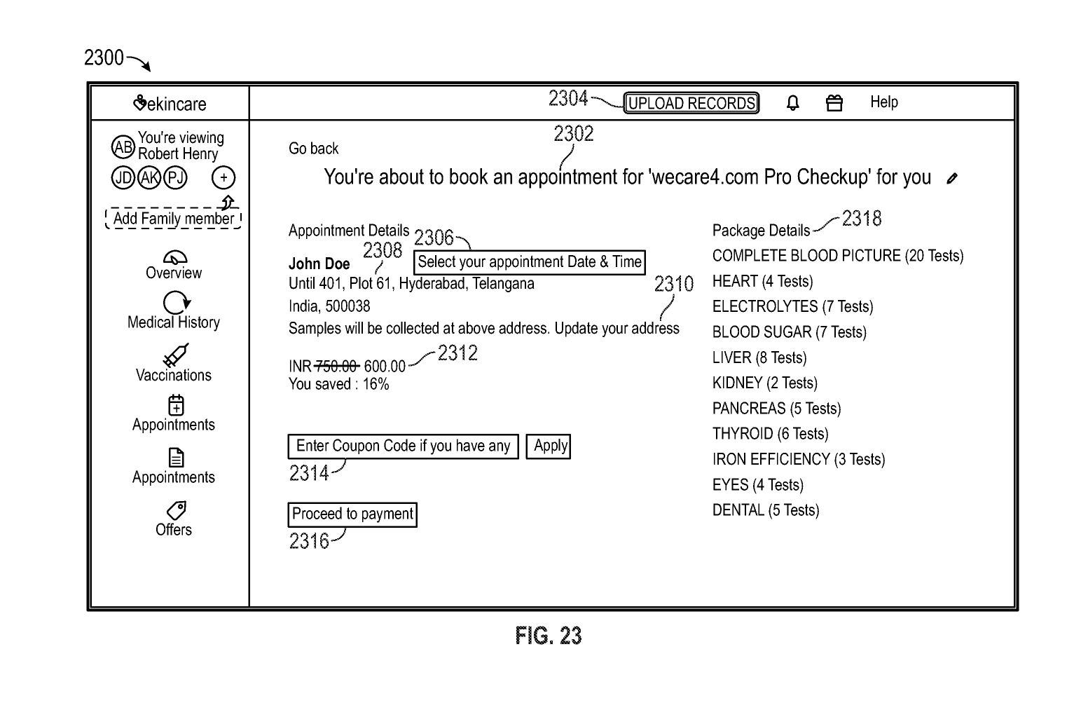

Referring to FIG. 23 is a representation 2300, of the payment procedure to be adapted for finalizing an appointment, according to an exemplary embodiment of the present disclosure. An alert comment 2302 “you are about to book an appointment for pro check-up for you” is displayed. An option to upload the records 2304 is provided for the individual who would enable the individual as well as the health care provider to have a ready access to the health records as and when required. An address 2308 is fed for adequate communication where an option to collect the samples 2310 may be provided. The fee applicable for the tests recommended along with the discount 2312 is displayed. A coupon code 2314 may be applied. A proceed to payment option 2316 enables the individual to go ahead with the payment formalities to fortify the appointment. A side-line view 2318 of the packages available may be displayed for an individual's convenience.

Although the present disclosure has been described in terms of certain preferred embodiments and illustrations thereof, other embodiments and modifications to preferred embodiments may be possible that are within the principles and spirit of the invention. The above descriptions and figures are therefore to be regarded as illustrative and not restrictive.

Thus the scope of the present disclosure is defined by the appended claims and includes both combinations and sub combinations of the various features described herein above as well as variations and modifications thereof, which would occur to persons skilled in the art upon reading the foregoing description.

What is claimed is:

1. A system for preventive health management for assessing an individual's health through consolidation and digitization of medical records comprising:
a medical history consolidating module configured to consolidate medical records of an individual in a digital format;
an assessment module configured to analyse medical records of an individual; and
a display module configured for interactively displaying a consolidated medical records of the individual in response to analysing the medical records, whereby the interactive displaying of health records enables an individual to self-learn and self-understand health profile.

2. The system of claim 1, wherein a timeline module configured to provide with a timeline of health checks in response to analysing the medical records and keep a track of health checks.

3. The system of claim 1, wherein a document module configured to aggregate medical records uploaded by the individual in online mode and offline mode.

4. The system of claim 1, wherein a reminder module configured to generate a reminder regarding at least one of: pending health check-ups; and upcoming health check-ups; pending medications; upcoming medications; pending immunizations; and upcoming immunizations;

5. The system of claim 1, wherein a profile module enables an individual to at least one of: update personal health profile; update family health profile.

6. The system of claim 1, wherein an offer configured to display one or more offerings related to health check-ups; medications; and immunizations.

7. The system of claim 1, wherein a personalized access provider module configured to provide a personalized health care access through one or more healthcare service providers in response to analysing a chronic health condition of the individual.

8. The system of claim 1, wherein an appointment engine module configured to provide a self-check or self-service by seeking an appointment for various health tests to be conducted under certain predetermined circumstances in a nearby diagnostic center with a prepaid mode.

9. A system for assessing an individual's health through consolidation and digitization of medical records comprising of:
a medical history consolidating module configured to consolidate medical records of one or more individuals associated to an organisation in a digital format;
an assessment module configured to analyse medical records of one or more individuals; and
an aggregating module enabled to provide an aggregate of a medical records of one or more individuals, whereby the aggregation enables an organization and one or more individuals to self-learn and self-understand health profile of one more individuals.

10. A method for preventive health management for assessing an individual's health through consolidation and digitization of medical records comprising:
consolidating a medical records of an individual in a digital format;
analyse medical records of an individual; and
displaying a consolidated medical records of the individual in response to analysing the medical records, whereby the interactive displaying of health records enables an individual to self-learn and self-understand health profile.

11. A method for preventive health management for assessing an individual's health through consolidation and digitization of medical records comprising: displaying consolidated medical records of the individual in response to analysing the medical records, whereby the interactive displaying of health records enables an individual to self-learn and self-understand health profile.
